(19) United States
(12) Patent Application Publication
(10) Pub. No.: US 2018/0289878 A1
(43) Pub. Date: Oct. 11, 2018

(54) HEART PUMP ARRANGEMENT, AND HEART PUMP ARRANGEMENT SYSTEM

(71) Applicant: Berlin Heart GmbH, Berlin (DE)

(72) Inventors: Kim Peter Winterwerber, Berlin (DE); Helge Krambeck, Berlin (DE); Sebastian Kallenbach, Kassel (DE); Daniel Strommenger, Berlin (DE)

(73) Assignee: Berlin Heart GmbH, Berlin (DE)

(21) Appl. No.: 15/570,722

(22) PCT Filed: May 4, 2016
(86) PCT No.: PCT/EP2016/060096

§ 371 Date: Oct. 30, 2017

(30) Foreign Application Priority Data
May 6, 2015 (EP) 15166554.4
Jun. 9, 2015 (EP) 15171285.8

(51) Int. Cl. A61M 1/12; A61M 1/10

(52) U.S. Cl. CPC A61M 1/127; A61M 1/1086; A61M 2205/8262; A61M 2205/8206; A61M 1/122

(57) ABSTRACT

A heart pump arrangement including a heart pump and a control apparatus connected thereto, where the control apparatus has a first connector element of a first connection device, and including an intermediate plug-in module, which has a second connector element, which is detachably connectable to the first connector element, wherein the intermediate plug-in module has a second connection device, which is non-detachable or which is detachable within a framework of a second connection, for connection to an electrical power source, and additionally a third connection device, which is non-detachable or which is detachable within a framework of a third connection, for connection to a data processing device, in particular a terminal. By means of the connection possibilities of the intermediate plug-in module on the secondary side, incorrect connections to connected batteries, power supply units, or other apparatuses to be connected are minimised.

### CROSS-REFERENCE TO RELATED APPLICATIONS

This application is a 371 nationalization of international patent application PCT/EP2016/060096 filed May 4, 2016, the entire contents of which are hereby incorporated by reference, which in turn claims priority under 35 USC § 119 to European patent application EP 15 166 554.4 filed on May 6, 2015, and European patent application EP 15 171 285.8 filed on Jun. 9, 2015.

### TECHNICAL FIELD

The invention lies in the field of electrical engineering and can be used particularly advantageously in the field of medical technology. In particular, the invention relates to heart pump arrangements and corresponding control apparatuses and peripheral equipment.

### BRIEF DESCRIPTION OF THE DRAWINGS

FIG. 1 shows a schematic view of a patient's body in section with an implanted heart pump and a control apparatus;

FIG. 2 schematically shows a control apparatus with an intermediate plug-in module;

FIG. 3 shows three different combinations of an intermediate plug-in module with one or more power sources connected thereto;

FIG. 4 schematically shows a multiplexer;

FIG. 5 shows the detailed structure of a control apparatus; and

FIG. 6 shows a variant of a control device of a control apparatus.

### DETAILED DESCRIPTION

For some time, implantable heart pumps for assisting and partially replacing the heart function of patients have been known, which for example can be operated as VAD systems (ventricular assist devices). Implantable pumps of this type are usually operated by control units or control apparatuses located outside the patient's body, which are connected by means of percutaneous cables to the actual implanted pump.

Electrical power sources, such as batteries (secondary cells or primary cells), but also appropriate power supply units, are usually provided for the operation of pumps of this kind and are connected to the external control apparatus of the pump. Electrical power sources of this kind are exchangeable, for example so as to be able to change batteries or so as to be able to adapt the operation to the power supply systems currently available. The external control apparatus exchanges information with the implanted elements, that is to say for example with the heart pump itself. This information relates for example to parameters for setting the operation of the pump, measurement data regarding the operational behaviour of the pump, or patient-related data. In order to set operating parameters of the pump or in order to analyse operating data by the treating doctor, a data connection is usually established between a control apparatus and an external apparatus, i.e. a data processing device—also referred to as a terminal in this context. In addition, the data can be read from the control apparatus, or data can also be stored in the control apparatus.

During the period of data exchange or the period of exchange of an external power source during maintenance work by the treating doctor, the power supply to the heart pump arrangement must be ensured. In the case of maintenance work, there are thus often a number of apparatuses to be connected to the control apparatus, which increases the risk of operating and usage errors and the connection of incorrect or faulty components. A particularly great risk lies in the separation of the system from the power supply. Indeed, a redundancy of the power supply is provided in principle, however the risk of total failure increases with disconnection from a power supply source and fallback to a reserve level.

Against the background of the prior art, the object of the present invention is therefore to create a heart pump arrangement with which operating errors are minimised and a failure of parts of the system has minimal consequences.

Accordingly, the object according to the present invention is achieved by a heart pump arrangement comprising a heart pump and a control apparatus connected thereto, which control apparatus has a first connector element of a first connection device, and comprising an intermediate plug-in module, which has a second connector element, which is detachably connectable to the control apparatus (in some variants to the first connector element), wherein the intermediate plug-in module has a second connection device, which is non-detachable or which is detachable within a framework of a second connection, for connection to an electrical power source, in particular a power supply unit or a battery, and/or additionally a third connection device which, is nondetachable or which is detachable within a framework of a third connection, for connection to a data processing device, in particular a terminal.

A heart pump of this kind is often implanted and connected to a control apparatus by means of a percutaneous line connection. However, the pump can also be arranged outside the patient's body and connected by means of catheters to elements of the circulatory system in order to remove and feed blood. A connection device can usually be provided within the context of this connection between the heart pump and the control apparatus.

In addition, a first connection device is provided, by means of which the intermediate plug-in module can be plugged into the control apparatus. The intermediate plug-in module for its part has a second connection device for connection to an electrical power source, and a third connection device for connection to a data processing device, in particular a terminal. The intermediate plug-in module is thus connected to the control apparatus only by means of a single connector element and for its part offers two connection devices for an electrical power source and a data processing device, wherein each of these can be formed individually or both as connection devices. Either the power supply module or the data processing device, or both, can be non-detachably connected to the intermediate plug-in module, or separate connection devices are available on the intermediate plug-in module for one or both of these two external elements. Corresponding connection devices can be designed in such a way that, for example, it is not possible to confuse the insertion slots, and therefore each connector element, for example of an electrical power source or a data processing device, can only be plugged into a single connector element on the intermediate plug-in module. Mix-ups and incorrect connections are therefore precluded.

In the prior art, a control apparatus is usually provided with a conventional connection device which has a connector element which can be connected to a single connector element of peripheral equipment. In this regard, the plugging of the connection to a data processing device into the plug-in connection was associated in the past with the risk of the heart pump arrangement being completely separated from an external power supply. In the prior art, it could also occur that the electrical power source might be plugged mistakenly into a data connection.

By branching the connector points used for different purposes within the intermediate plug-in module to two separate connections, mix-ups are precluded. Incorrect connections can be further avoided by a different shaping of the connection devices accordingly.

Here, the connector element can be, for example, a plug-in connection element, an optical connection element, and/or a wireless, radio-based connection element. The connector element can thus be connected both as a physical connector element and as a virtual connector element for the transmission of data from the control apparatus to a further interface connected to the intermediate plug-in module, such as an external programmer or an external readout and/or maintenance module. In a further embodiment the connection device is designed for inductive coupling. The corresponding connector element can be formed for example as a coil, which is arranged in such a way that a corresponding coil can be designed for maximum inductive coupling. Here, the two coils can be oriented concentrically with one another, for example.

An advantageous embodiment of the invention provides that the control apparatus is connected by means of a non-wired connection, in particular a radio link, to the data processing device, in particular the terminal. A multi-channel connection between the control apparatus and the data processing device or a terminal is thus produced and ensures an increased availability by redundancy. The non-wired connection can function here by means of conventional WLAN or Bluetooth standards or also in accordance with other communication standards, and can be the connection used primarily or also the connection used secondarily.

A further advantageous embodiment of the invention, as already mentioned above, provides that the intermediate plug-in module has separate connector elements for each of the second and the third connection device. In this way the plug-in connectors of the second and third plug-in connection can be designed geometrically differently, so that a mix-up or even the actual plugging itself of an incorrect plug-in connector into or at an incorrect plug-in connector provided on the intermediate plug-in module is made impossible.

A further advantageous embodiment of the invention can provide that the intermediate plug-in module is connected by means of a second connection device to an electrical power source, wherein a connector element of the third connection device branches off from the line between the intermediate plug-in module and the power supply assembly. In this case, it is suggested to the user of the intermediate plug-in module to firstly connect the second connection device and thus connect the intermediate plug-in module to an electrical power source and to only then establish the third plug-in connection by plugging a corresponding plug-in connector into the connector element provided on the line between the intermediate plug-in module and the electrical power source. It is thus ensured to a high degree that when establishing the connection between the control apparatus and a data processing device/a terminal it is ensured that the control apparatus is already permanently connected to a power supply assembly.

A further advantageous embodiment of the invention provides that the control apparatus has a recess into which the intermediate plug-in module can be inserted and in which there is arranged a first connector element of the first connection device. The recess can be shape-coded and for example can be formed in the manner of a blind bore. The recess can be cube-shaped or generally can be triangular, square or polygonal in cross-section. The shape can be complementary to the shape of the intermediate plug-in module. By means of the shape of the recess, it can be ensured that only corresponding intermediate plug-in modules, which have a matching shape, can be plugged into the connector element of the first connection device. The shape of the recess can also be matched for example to the shape of a battery, as electrical power source, which can be fitted on the same connection device.

It can additionally be provided advantageously that at least one electrical plug contact, in particular two electrical plug contacts of the first connector element is/are provided both for the connection of the control apparatus to a power supply source and for the connection to a data processing device.

When connecting the control apparatus to an electrical power source, individual pins of a line connection are usually used already both for the power transmission and for data transmission. To this end, an I²C bus for example is used in order to separate the functions so as to be able to read information from a battery. Conversely, on the battery side, the corresponding connection points can be used to identify the establishment of contact/connection.

The connection conductors available in the first connection device can also be used at the same time for the transmission of data from and to an electrical power source and for the connection of the control apparatus to a data processing device/a terminal, and can also be used there for the exchange of data. In addition, the same pins, or at least some pins of the connection device can be used for the transmission of electrical energy.

Here, it can be provided advantageously that the intermediate plug-in module can be connected to the control apparatus by means of a data bus connection.

In order to be able to manage the wide range of connection functions of the first connection device, it can be provided advantageously that the intermediate plug-in module has a multiplexing device, in particular a time-multiplexing device, for connection of the first connection device alternately to the second and third connection device. The multiplexing device can thus be switched over periodically between a connection of the power supply source via the intermediate plug to the control apparatus on the one hand, and a connection of a data processing device/a terminal via the intermediate plug-in module to the control apparatus on the other hand. These two connections can thus be decoupled.

In a further embodiment the control apparatus is fixedly provided with the connected power source, in particular a battery.

The invention also relates to a heart pump arrangement of the above-described type, and additionally also to a heart pump arrangement system comprising a heart pump arrangement according to any one of claims 1 to 11 and comprising an energy storage device, which, instead of the intermediate plug-in module, can be detachably connected to the first connector element. In a way that is advantageous for a patient, the control apparatus can thus be connected in most cases to an electrical power source; for example, a battery can be plugged into a recess on the control apparatus, whereas at certain times, for example for maintenance, an intermediate plug-in module is plugged in instead of the energy storage device. For the patient, the heart pump arrangement system can be provided as a set with the intermediate plug-in module and the energy storage device.

In a further aspect, the present application relates to a control apparatus for a heart pump arrangement. Here, the control apparatus comprises a control device for controlling a heart pump and a housing with a connection device which has exactly one connector element, for example a plug-in connector element or another of the connector elements described above, for detachably connecting the control apparatus to an intermediate plug-in module. A control apparatus of this type, besides a potential connection device to the heart pump itself, comprises merely precisely one connector element for an intermediate plug-in module. Here, the connector element can be, for example, a plug-in connection element, an optical connection element and/or a wireless, radio-based connection element. The connector element can thus be connected both as physical connector element and as purely virtual connector element for transmission of data from the control apparatus to a further interface connected to the intermediate plug-in module, such as an external programmer or an external readout and/or maintenance module.

The control apparatus thus has merely a single connector element. It is thus possible to dispense with further connector elements, which drastically reduces the likelihood of an operating error or incorrect connection of the control apparatus to an intermediate plug-in module. The exactly one connector element is arranged here within the housing or on an outside of the housing.

In a further embodiment, a power source, in particular a battery, fixedly connected to the control apparatus is arranged within the housing. This “internal” power source is used for example to serve as a temporary replacement for an external power supply, which for example is to be exchanged. In addition, a power supply during a failure of the connector element or the external power source is avoided.

In a further embodiment the connection device of the control apparatus comprises a switching unit connected to the connector element, which switching unit is configured in such a way that control signals both of an external power source and control signals of an external user terminal can be processed by the control device. For example, it is hereby ensured that both pure power sources (without further data exchange), which are connected to the control apparatus via the precisely one connector element, and pure data analysis units, which provide merely data, but no power necessary for operation of the heart pump, can be coupled to the connector element. The switching unit can comprise for example a communication interface, which connects a microcontroller arranged in the control apparatus, a processor or a field programmable array alternately to the control signals of an external power source, for example a battery, and an external data processing device. Here, in some exemplary embodiments, the switching unit can determine whether the control signals arriving at the connector element originate from a power source and/or a further data processing device or whether control signals of the control apparatus are intended for an external power source or a further data processing device. Alternatively, a transmission protocol can be selected which switches at fixed time intervals between a possibly provided power source and a possibly provided data processing device.

The intermediate plug-in module can comprise a housing and can be formed on the whole in a rigid manner, that is to say without flexible elements, such as cables. For example, the intermediate plug-module has a plug-in connector element facing the control apparatus, which plug-in connector element is integrated in the housing, and for example additionally one or more plug-in connector elements on the side of the intermediate plug-in module facing away from the control apparatus in the plugged-in-state, which plug-in connector element(s) is/are likewise integrated in the housing. They are accessible from the outside when the intermediate plug-in module is inserted into the control apparatus.

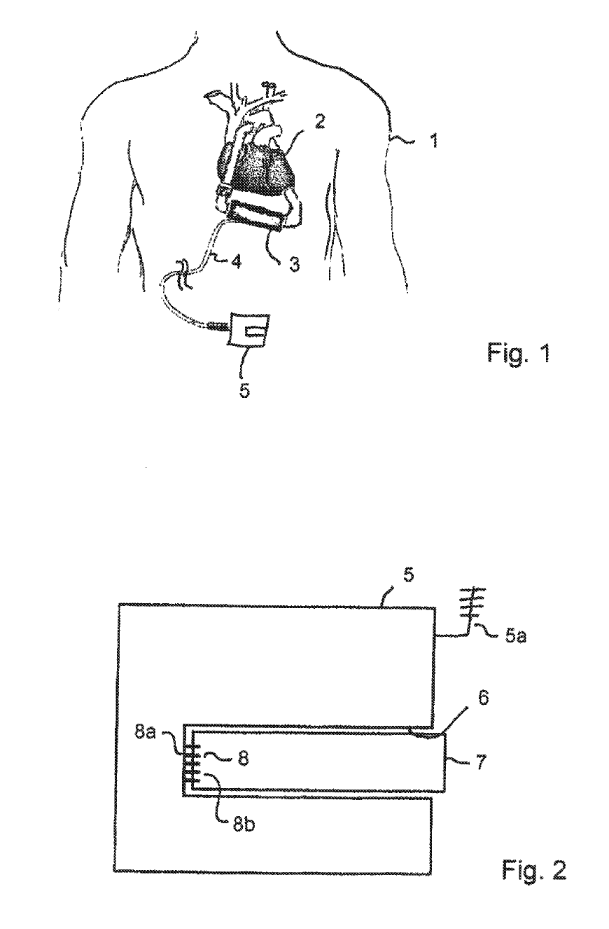

The invention will be presented hereinafter on the basis of exemplary embodiments in figures of a drawing and then described. In the drawing FIG. 1 shows a view of a patient's body 1 with a patient's heart 2, to which a heart pump 3 is connected as VAD (ventricular assist device). The heart pump 3 is implanted in the patient's body and is connected transcutaneously by means of a connection line 4 to a control apparatus 5 outside the patient's body. Both data, for example pump parameters, and electrical energy are exchanged with the heart pump 3 via the control apparatus 5.

In FIG. 2 a control apparatus is schematically illustrated in an enlarged manner, wherein an intermediate plug-in module 7 is inserted into a recess 6 of the control apparatus 5. The intermediate plug-in module 7 is connected to the control apparatus 5 by means of a first connection device 8. The first connection device 8 provides a first connector element 8a on the control apparatus 5 and a second connector element 8b on the intermediate plug-in module 7. Here, the first connector element 8a can be a plug, and the second connector element 8b can be a socket, or vice versa, or the connector elements 8a and 8b can each comprise a plug or a socket, or a number of mixed contacts can also be provided on both sides of the first connection device, wherein both plug elements and sockets are provided on both sides.

What is important in this regard is that the first connection device 8 can be plugged in and detached without difficulty. The actual connector elements 8a, 8b are protected here in the recess 6 of the control apparatus 5.

In principle, the invention can be used with greatest advantage in implanted heart pumps, however it can also be used with non-implanted artificial hearts arranged outside the patient's body and connected to a patient's circulatory system via cannulas.

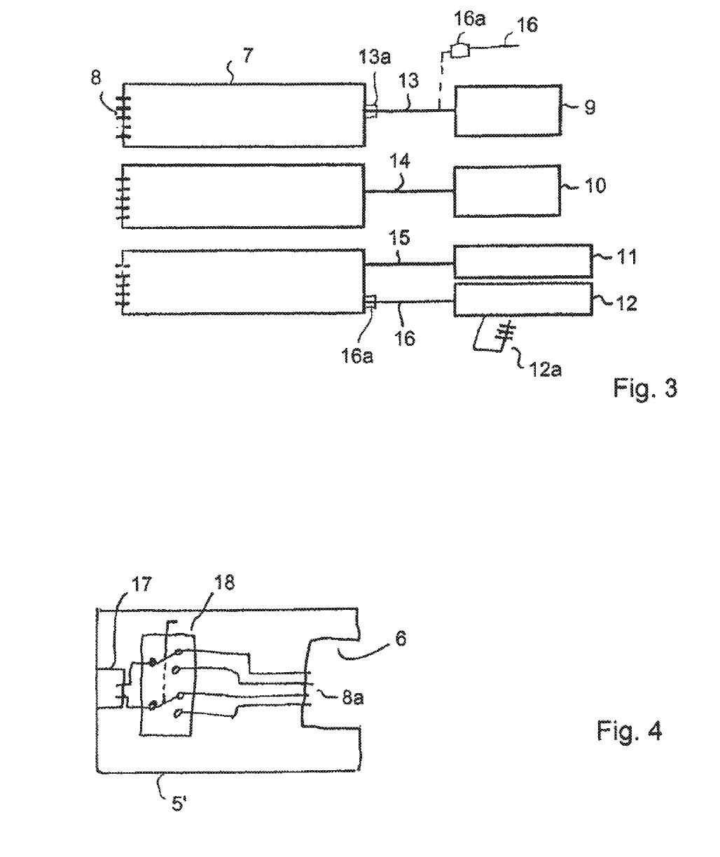

In FIG. 3 it is illustrated that the intermediate plug-in module can be connected on the secondary side, i.e. with respect to the side facing away from the control apparatus 5 and the first connection device 8, to different further elements and/or power sources. In FIG. 3 the intermediate plug-in module 7 is illustrated in three variants, wherein in the uppermost variant the intermediate plug-module is connected to a mains power unit 9, whereas in the middle variant the intermediate plug-in module is connected to a power supply source in the form of a battery 10. In the variant shown at the bottom, the intermediate plug-in module is connected both to a power supply element 11, either in the form of a battery or in the form of a power supply unit, and to a data processing device 12 in the form of a terminal. Lines 13, 14, 15 and 16 are shown respectively for connection of the individual power sources 9, 10, 11 and 12, which lines connect the individual elements to the intermediate plug-in module 7. Here, each of the lines 13, 14, 15, 16 can be connected in principle by means of a connection device 13a, 16a to the intermediate plug-in module 7. For example, the connection device can be formed in each case as a plug-in connection device. However, a further variant can also be that the line 15 fixedly and non-detachably connects the intermediate plug-in module to the power supply source 11, whereas the data processing device/the terminal 12 can be connected to the intermediate plug-in module by means of a pluggable line 16 within a framework of a third connection device. By means of this configuration, a permanent connection of the control apparatus to a power supply is ensured.

Here, it can also be provided advantageously that, for the case in which both the line 15 and the line 16 can be connected to the intermediate plug-in module by means of a connection device, the corresponding connector elements are formed differently, in such a way that it is impossible for them to be mixed up at the time of being plugged in.

The data processing device 12 can also be equipped with a transceiver device 12a for a radio link in order to be coupled to the control apparatus additionally via a radio link, parallel to the wired communication.

FIG. 4 schematically shows an illustration of a control apparatus 5′ for a heart pump arrangement according to the invention with a recess 6 in which a connector element 8a is arranged.

The so-called system is illustrated symbolically inside the control apparatus 5′ and is denoted by reference sign 17. All functional elements of the control apparatus that are used to control the heart pump 3, and memory devices for measured values and pump parameters are combined in the system. A multiplexing device 18 is shown between the system 17 and the connector element 8a and operates different connection points of the connector element alternately in accordance with a time-multiplexing regime with different communication protocols. Different apparatuses which communicate by means of different bus systems and communication protocols can thus be connected to the different pins/contacts of the first connection device 8. Inside the intermediate plug-in module 7, the number of lines connected to the connector element 8b can thus be divided into different groups, which lead on the secondary side of the intermediate plug-in module 7 to different lines and/or connector elements. There, different apparatuses, such as power supply units, batteries/battery packs or a data processing device, for example in the form of a terminal, can then be connected to the different connector elements/lines.

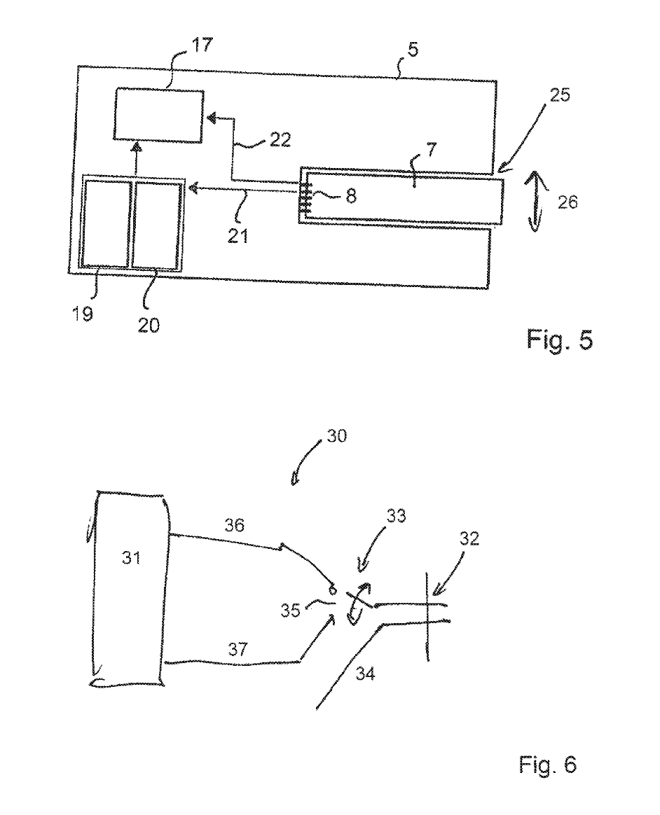

In FIG. 5 a control apparatus 5 with a system part 17 is illustrated schematically, in which the functional elements of the control apparatus, such as a processor control system for the pump, memory devices for pump parameters and the like and the monitoring of the power supply, the state of charge of the batteries, etc., are accommodated. The system 17 is connected on the one hand to the unit with two power supply sources 19, 20, wherein these constitute internal batteries, which can supply power, respectively, to the system and the heart pump for approximately fifteen minutes, for example whilst an external battery is swapped or an external power supply unit is connected or exchanged.

On the other hand, the system 17 is connected to the first connection device 8 and via this to the intermediate plug-in module 7. If the connection to an external battery or a mains power unit is established via the intermediate plug-in module 7, each of the two internal batteries 19, 20 can thus be charged via the charging line 21. By means of an external power supply source, the system 17 and the heart pump directly connected thereto can additionally be supplied with power via the further line 22.

In principle, the connection lines 13, 14, 15, 16 are formed at least optionally as data bus connections, i.e. the connection 16 between the intermediate plug-in module 7 and a data processing device can be a pure data bus, whereas the other lines 13, 14, 15 should at least also allow a feed voltage or a feed current to pass through as power supply parallel to a data traffic protocol.

In addition and parallel to the above-describe connections, a non-wired connection, for example in the form of a radio link, can exist as primary connection between the control apparatus 5 and the data processing device 12, and is indicated by the antennas 12a in FIGS. 3 and 5a in FIG. 2. The wired connection 16 then forms a fallback level for the case in which the non-wired connection fails or is faulty.

On the basis of FIG. 5, it can be seen that the control apparatus comprises merely precisely one connector element 8 for an intermediate module. In a variant, further connector elements are not provided. The connector element 8 can be arranged for example in a recess 25 of the housing (indicated schematically by the line marked as a control apparatus 5). An intermediate module can also have a housing, the external dimensions of which are such that they are received in the recess 25, preferably in a form-fitting manner, so that the precisely one connector element 8 of the control apparatus can be coupled to the connector element of the intermediate module. The connector elements of the control apparatus and of the intermediate module are formed in a manner complementary to one another; in the present case the connector element is a plug-in connection element.

The intermediate plug-in module is for example formed in a manner complementary to the recess in the housing of the control apparatus, in such a way that more than half of the volume of the intermediate plug-in module, preferably more than 70%, more preferably more than 90%, is received in the recess.

The shape and size of the intermediate plug-in module can be designed in such a way that the intermediate plug-in module is received substantially in the recess of the control apparatus and the intermediate plug-in module is flush with with the contour of the control apparatus. It is then advantageous to provide a handling device, for example in the form of an eyelet, a tab or an undercut, with the aid of which the intermediate plug-module can be removed again from the recess.

The intermediate plug-in module is advantageously cableless, in other words it has no protruding cables connected permanently and non-detachably to the module, and instead has exclusively plug-in connector elements.

In a particular embodiment the intermediate plug-in module can be designed in a manner matching the recess in the control apparatus, in such a way that it can be plugged into said recess in a number of defined different positions, in particular two positions, in each of which different electrical plug-in connections to the control apparatus are established. The intermediate plug-in module can thus perform different functions in different plug-in positions, in particular can establish different electrical connections.

By means of the recess 25 schematically indicated in FIG. 5, it is additionally ensured that the housing of the control apparatus protects the intermediate module from being accidentally pulled out in a direction 26, since here the inner walls of the recess protect the intermediate module against greater movements. The recess can also be referred to as a port, on the wall of which there is arranged a connector element for connection to a connector element of the intermediate module, the connector element preferably being arranged on the base of the port or the recess.

FIG. 6 shows a variant of a control device of the control apparatus. The control device 30 comprises a microcontroller 31, which is coupled to a connection device 32. The connection device for example comprises a plug-in connector element 32a, which can be coupled to a corresponding plug-in connector element of an intermediate module. Since the control apparatus in the present example comprises exactly one connector element for an intermediate module, the embodiment of the connection device 32 on the control apparatus side has both a circuit 33 for communication signals or control signals and a circuit 34 for power, i.e. a circuit which makes power available to the pump from a power source (power supply unit or battery) connected to the intermediate module and/or from an internal power source of the control apparatus. The circuit 33 is coupled to the microcontroller 31 and, besides a switching element 35, comprises a circuit 36 for control signals of a power source, for example a battery or a mains power unit, and a circuit 37 for control signals of a user terminal, which can be coupled to the control apparatus via the intermediate module. Here, the switching element 33 can alternately couple the circuit 36 or the circuit 37 to the connection device 32, so that the microcontroller can process both control signals of a power source and of the user terminal. Here, the microcontroller is formed in such a way that, for example regardless of the coupled intermediate module, control signals of the user terminal or of the power source can be processed and/or can be sent to the intermediate module. An internal power source (not shown) of the control apparatus can be charged via the circuit 34, for example provided the microcontroller receives corresponding control signals of the circuit 37. In other exemplary embodiments, other methods known to a person skilled in the art for control signal transmission can be selected.

In the present example, the intermediate module can connect a power source or a user terminal or both to the control apparatus. In this way, the precisely one connection device is not a limitation of the connectivity of the control apparatus. Rather, the connection of the intermediate module to the control apparatus is significantly simplified for the user, and application safety is increased. The identification of the intermediate module and the processing of the corresponding control signals are performed and/or controlled by the microcontroller 31. Of course, the microcontroller can be replaced for example by a processor or can be connected to a processor of the control apparatus.

By means of the invention described here, the susceptibility to errors and the risk of operating errors in a heart pump system are significantly reduced. Combination possibilities for the patient which increase patient comfort are provided.

1. A heart pump arrangement comprising a heart pump and a control apparatus connected thereto, wherein the control apparatus has a first connector element of a first connection device, and comprising an intermediate plug-in module, which has a second connector element, which is detachably connectable to the control apparatus, wherein the intermediate plug-in module has a second connection device, which is non-detachable or which is detachable within a framework of a second connection, for connection to an electrical power source, in particular a power supply unit or a battery, and/or a third connection device, which is non-detachable or which is detachable within a framework of a third connection, for connection to a data processing device, in particular a terminal.

2. The heart pump arrangement according to claim 1, wherein the control apparatus is connected by means of a non-wired connection, in particular a radio link, to the data processing device, in particular the terminal.

3. The heart pump arrangement according to claim 1, wherein the intermediate plug-in module has separate connector elements for each of the second and third connection device.

4. The heart pump arrangement according to claim 1, wherein the intermediate plug-in module is connected by means of a second connection device to an electrical power source, wherein a connector element of the third connection device is connected to the line between the intermediate plug-in module and the power supply assembly.

5. The heart pump arrangement according to claim 1, wherein the control apparatus has a recess, into which the intermediate plug-in module can be inserted and in which a first connector element of the first connection device is arranged.

6. The heart pump arrangement according to claim 1, wherein at least one electrical plug-in contact, in particular two electrical plug-in contacts, of the first connector element is/are provided both for the connection of the control apparatus to an electrical power source and for the connection to a data processing device.

7. The heart pump arrangement according to claim 1, wherein the intermediate plug-in module can be connected by means of a data bus connection to a data processing device.

8. The heart pump arrangement according to claim 1, wherein the intermediate plug-in module can be connected by means of a data bus connection to the control apparatus.

9. The heart pump arrangement according to claim 1, wherein the intermediate plug-in module has a multiplexing device, in particular a time-multiplexing device, for connecting the first connection device alternately to the second and third connection device.

10. The heart pump arrangement according to claim 1, wherein the control apparatus is provided with a power source, in particular a battery, fixedly connected thereto.

11. The heart pump arrangement according to claim 1, wherein the control apparatus has a recess which receives the intermediate plug-in module through its complementary shape, in such a way that at least 50% of the volume of a housing of the intermediate plug-in module, in particular more than 80%, more particularly more than 90% of the volume, is received in the recess.

12. The heart pump arrangement according to claim 1, wherein, in the plugged-in state, the intermediate plug-in module is flush with the outer contour of the control apparatus.

13. The heart pump arrangement according to claim 1, wherein all plug-in connection elements of the intermediate plug-in module are arranged at fixed angles to one another in a housing.

14. The heart pump arrangement according to claim 1, wherein a device for removing the intermediate plug-in module from the recess of the control apparatus is provided in the form of an undercut, a grip, a tab or an eyelet.

15. A heart pump arrangement system with a heart pump arrangement according to claim 1 and with an energy storage device, which, instead of the intermediate plug-in module, can be detachably connected to the first connector element.

16. A control apparatus for a heart pump arrangement, wherein the control apparatus comprises a control device for controlling a heart pump by means of a line and a housing with a connection device, and the connection device has precisely one first connector element for detachably connecting the control apparatus to an intermediate plug-in module.

17. The control apparatus according to claim 16, wherein a power source, in particular a battery, fixedly connected to the housing is arranged in said housing.

18. The control apparatus according to either one of claim 16, wherein the connection device comprises a switching unit connected to the first connector element, so that control signals both of an external power source and control signals of an external user terminal can be processed by the control device.
